(19) United States
(12) Patent Application Publication
(10) Pub. No.: US 2016/0143680 A1
(43) Pub. Date: May 26, 2016

(54) CEMENT CARTRIDGE DISPENSING DEVICE HAVING ROLLING CLAMPING BODIES

(71) Applicant: Heraeus Medical GmbH, Wehrheim (DE)

(72) Inventors: Clemens GREINER, Garbsen (DE); Sebastian VOGT, Erfurt (DE)

(73) Assignee: HERAEUS MEDICAL GMBH, Wehrheim (DE)

(21) Appl. No.: 14/934,610

(22) Filed: Nov. 6, 2015

(30) Foreign Application Priority Data
Nov. 25, 2014 (DE) 10 2014 117 224.8

(51) Int. Cl. A61B 17/88; B05C 17/01; B05C 17/005

(52) U.S. Cl. CPC A61B 17/8819; A61B 17/8825; B05C 17/00596; B05C 17/0116

(57) ABSTRACT

Dispensing device for cement cartridges for polymethylmethacrylate bone cements comprising at least one manually actuatable rocker lever, an adapter for a cement cartridge, a clamping rod that can be propelled in the direction of the adapter, at least one propulsion body shiftable in axial direction with respect to the axis of the clamping rod, at least one sphere-shaped or roller-shaped clamping body arranged in a cavity of the at least one propulsion body, an elastic element which pushes the at least one clamping body of the at least one propulsion body from the direction of the adapter onto an inclined surface or edge of the cavity of the at least one propulsion body, a spring element which pushes onto the at least one propulsion body opposite to the propulsion direction of the clamping rod, and at least one securing element which touches against the clamping rod.

The invention relates to a dispensing device for cement cartridges for polymethylmethacrylate bone cements comprising at least one manually actuatable rocker lever and one adapter for a cement cartridge.

Accordingly, the subject matter of the invention is a manually driven device for dispensing polymethylmethacrylate bone cement dough from cartridges of vacuum cementing systems. The device is intended for single use only.

### BACKGROUND OF THE INVENTION

Articular endoprostheses are used commonly in orthopaedics and trauma surgery to replace human joints that have been damaged by disease, accident or wear. In this context, permanent mechanical fixation of articular endoprostheses is effected by mechanical clamping (press-fit) or by cementing using polymethylmethacrylate bone cements (PMMA bone cements).

PMMA bone cements consist of a liquid monomer component and a powder component. The monomer component generally contains the monomer, methylmethacrylate, and an activator (N,N-dimethyl-p-toluidine) dissolved therein. The powder component, also called bone cement powder, comprises one or more polymers that are produced through polymerisation, preferably suspension polymerisation, based on methylmethacrylate and co-monomers, such as styrene, methylacrylate or similar monomers, often a radiopaquer, and the initiator, dibenzoylperoxide. The powder component is mixed with the liquid monomer component before application of the cement During the mixing process, swelling of the polymers of the powder component in the methylmethacrylate generates a dough that can be shaped plastically and is the actual bone cement. In this context, the activator, N,N-dimethyl-p-toluidine, reacts with dibenzoylperoxide forming radicals in the process. The radicals thus formed trigger the radical polymerisation of the methylmethacrylate. Upon advancing polymerisation of the methylmethacrylate, the viscosity of the cement dough increases until the cement dough solidifies. The solidified polymethylmethacrylate bone cement is mechanically stable and can permanently attach articular endoprostheses in the bone tissue of the patients.

The components of polymethylmethacrylate bone cements (PMMA bone cements) can be mixed with spatulas in suitable mixing cups. One disadvantage of said procedure is that air inclusions may be present in the cement dough thus formed and can cause destabilisation of the cured bone cement later on. For this reason, it is preferred to mix bone cement powder and monomer liquid in vacuum mixing systems, since mixing in a vacuum removes air inclusions from the cement dough to a large extent and thus achieves optimal cement quality. Bone cements mixed in a vacuum have clearly reduced porosity and thus show improved mechanical properties. A large number of vacuum cementing systems have been disclosed of which the following shall be listed for exemplary purposes: U.S. Pat. No. 6,033,105 A, U.S. Pat. No. 5,624,184 A, U.S. Pat. No. 4,671,263 A, U.S. Pat. No. 4,973,168 A, U.S. Pat. No. 5,100,241 A, WO 99/67015 A1, EP 1 020 167 B1, U.S. Pat. No. 5,586,821 A, EP 1 016 452 A2, DE 36 40 279 C2, WO 94/26403 A1, EP 1 005 901 A2, U.S. Pat. No. 5,344,232 A.

Cementing systems, in which both the cement powder and the monomer liquid are already packed in separate compartments of the mixing systems and are mixed with each other in the cementing system only right before application of the cement, are a development of cementing technology. Such full-prepacked mixing systems have been proposed in the patents, EP 0 692 229 B1, DE 10 2009 031 178 B3, EP 0 875 456 B3, U.S. Pat. No. 6,709,149 B1, and EP 1 140 234 B1 as well as U.S. Pat. No. 5,588,745 A.

In vacuum cementing systems, it is necessary to expel the cement dough from the cartridges by moving a plunger in order to apply the cement dough. Manual dispensing devices have been developed for this purpose.

EP 0 326 551 A1 prescribed an interesting lever system for manually driven devices. The rationale of said lever system is based on utilising a lever parallelogram to make optimally use of the force of the strongest fingers of the human hand, namely the index finger and middle finger, which is in contrast to a simple rocker lever.

The simplest dispensing devices are based on clamping rods having a tiltable small metal plate arranged on them that gets tilted by an asymmetrically engaging spring and thus clamps the clamping rod. The tiltable small metal plate is pressed in propulsion direction by a lever upon manual actuation, whereby the clamping small metal plate takes the clamping rod along with it. Subsequently, a spring pushes the small metal plate back into its starting position. This process is repeated until the clamping rod has pressed the conveying plunger of the vacuum cementing system sufficiently far in the direction of the cartridge head such that the desired amount of bone cement is pressed from the cartridge.

These devices are disadvantageous in that a retrograde motion due to the small metal plate sliding on the clamping rod is always possible during the clamping process. Due to the small metal plate sliding backwards, the user needs significantly more hand motions for extruding the bone cement dough than actually needed.

A further development consists of dispensing devices that use gear racks instead of simple clamping rods. One pertinent example is the dispensing device according to FIG. 28 of patent application US 2013 090 661 A1. It is an advantage of said devices that retrograde motion is basically excluded. However, the complex, laborious mechanics rendering the utilisation of said device for single use only questionable due to the relatively high production costs is disadvantageous.

It is the object of the invention to overcome the disadvantages of the prior art. Specifically, a dispensing device that is easy to manufacture can be used to manually extrude a polymethylmethacrylate on cement dough from cartridges of vacuum cementing systems is to be provided. The manually driven device shall be suitable and intended for single use only. The dispensing device is to consist largely of inexpensive plastic parts that can be manufactured by plastics injection moulding and few metal elements. During manual actuation of the dispensing device, a retrograde motion of the conveying plunger of the cartridge during the extrusion process due to elastic restoring forces of the plastic cartridge and the cement dough is to be prevented to the extent possible. The dispensing device to be developed is to enable just a single dispensation of polymethylmethacrylate bone cement dough. Reuse and the ensuing need for re-sterilisation of the dispensing device shall be excluded by the design.

### SUMMARY OF THE INVENTION

The objects of the invention are met by a dispensing device for cement cartridges for polymethylmethacrylate bone cements comprising at least one manually actuatable rocker lever, an adapter for a cement cartridge, and a clamping rod that can be propelled in the direction of the adapter, whereby at least one propulsion body is arranged to be touching against the clamping rod and is shiftable in axial direction with respect to the axis of the clamping rod, whereby the at least one propulsion body touching against the clamping rod forms at least one cavity together with the clamping rod, whereby the cavity comprises a surface or edge that is inclined in the propulsion direction of the clamping rod such that the cavity widens in the direction of the adapter in the region of said surface or edge, whereby at least one sphere-shaped or roller-shaped clamping body is arranged in the cavity of the at least one propulsion body, whereby the at least one clamping body of the at least one propulsion body touches the clamping rod in at least one spot or along a line, whereby an elastic element pushes the at least one clamping body of the at least one propulsion body from the direction of the adapter onto the inclined surface or edge of the cavity of the at least one propulsion body, whereby the rocker lever is supported as in a bearing such that it can rotate with respect to the clamping rod, such that, upon actuation of the rocker lever, an end of the rocker lever pushes onto the at least one axially shiftable propulsion body in propulsion direction of the clamping rod, whereby a spring element pushes onto the at least one propulsion body opposite to the propulsion direction of the clamping rod, at least one securing element touches against the clamping rod against which the propulsion body is supported such as to be mobile, whereby the at least one securing element touching against the clamping rod forms at least one cavity together with the clamping rod, whereby the cavity comprises a surface or edge that is inclined with respect to the propulsion direction of the clamping rod such that the cavity widens in the direction of the adapter in the region of said surface or edge, whereby at least one sphere-shaped or roller-shaped clamping body is arranged in the cavity of the at least one securing element, whereby the at least one clamping body of the at least one securing element touches the clamping rod in at least one spot or along a line, whereby an elastic element pushes the at least one clamping body of the at least one securing element from the direction of the adapter onto the inclined surface or edge of the cavity of the at least one securing element.

### DETAILED DESCRIPTION

Preferably, the at least one propulsion body touching against the clamping rod can be shifted in axial direction with respect to the axis of the clamping rod together with the clamping rod or can be shifted exclusively together with the clamping rod.

Presently, a rocker lever supported as in a bearing such as to be rotatable shall be understood to be a rocker lever that can be rotated by several degrees with respect to the clamping rod. Preferably, the rocker lever can be rotated by 20° to 70° with respect to the clamping rod.

Presently, a roller-shaped clamping body shall be understood to not only be a roller having cylindrical geometry. The roller shape can comprise the geometry of a multitude of different solids of rotation that are suitable for rolling on the clamping rod, which in turn depends on the shape of the clamping rod. A roller of this type can touch against the clamping rod in one spot (for example a sphere as clamping body on a cylindrical clamping rod) or in multiple spots (for example a roller in the shape of an hourglass as clamping body on a cylindrical clamping rod) or along a line (for example a cylindrical roller as clamping body on a 4-edged clamping rod) and can roll on the clamping rod over at least one spot or over the line.

It is theoretically feasible to use, instead of a clamping rod that can be propelled in the direction of the adapter, a clamping rod that can be propelled in working direction if the force arising from the propulsion of the clamping rod is redirected by joints, gear racks or the like, and if the working direction is changed thereby, and if the adapter for connection of the cement cartridge is situated in this location, and the clamping rod can therefore no longer be propelled geometrically in the direction of the adapter. This shall be understood as an equivalent design with the same working principle.

The present invention also proposes the dispensing device to comprise a housing with a handle, whereby the at least one securing element is preferred to be firmly connected to the housing and the at least one propulsion body is supported as in a bearing against the housing such as to be mobile.

By this means, the position of the at least one securing element with the housing is defined such that there is no need for separate storage of the securing element which simplifies the design of the dispensing device.

The invention can just as well provide the at least one propulsion body to surround the clamping rod, at least in regions thereof, preferably to surround it by at least 75%, particularly preferably to surround it completely.

By this means, the force can act on the clamping rod from all sides in a particularly simple manner. Moreover, this effectively prevents the dispensing device from being dismantled, such that said design is preferred.

In this context, the invention can provide the at least one propulsion body to comprise a feed-through that preferably is provided as a tube.

By this means, tipping and tilting of the propulsion body on the clamping rod can be prevented.

Alternatively, the invention can provide multiple axially shiftable propulsion bodies to touch against the clamping rod from different directions.

The individual propulsion body can thus be designed to be simpler. However, the assembly of the dispensing device is made more difficult and undesired dismantling of the dispensing device is made easier.

A refinement of the invention proposes the inclined surface to be shaped appropriately such that the at least one propulsion body and the at least one securing element comprises at least one cavity that is inclined, in longitudinal section, in the direction of the adapter, preferably comprises a wedge-shaped cavity, whereby the wedge shape is open in the direction of the adapter.

In this context, the invention can just as well provide the cavity to be open, in wedge shape, towards the propulsion direction of the clamping rod.

Preferred dispensing devices can also be characterised in that the at least one sphere-shaped or roller-shaped clamping body is arranged or supported as in a bearing such that it can rotate about at least one axis in the cavity of the at least one propulsion body and/or the at least one securing element.

A very effective and stable blockage of the clamping rod can be achieved by this means, since the rolling clamping bodies can tighten, depending on rolling direction, between the inclined surface or edge of the cavity and the clamping rod, and, during the reverse rotation, allow the rolling on the clamping rod such that the at least one propulsion body and/or the at least one securing element can slip over the clamping rod. In this context, even very small angles of rotation of the sphere-shaped or roller-shaped clamping body are sufficient to provide for the function of the clamping body. Preferably, the at least one sphere-shaped or roller-shaped clamping body can roll on the clamping rod by an angle of at least 1°.

According to a refinement of the dispensing device according to the invention, the invention can provide the cavity formed by the at least one propulsion body and the clamping rod and/or the cavity formed by the at least one securing element and the clamping rod is or are formed as a funnel, whereby the funnel surrounds the clamping rod.

By this means, the clamping bodies can wedge and/or lodge on all sides in the funnel and with the clamping rod and thereby block together with the clamping rod and/or clamp against the clamping rod.

Preferred embodiments can provide the clamping bodies to be made from ceramics or metal and particularly preferably to be made from steel and/or tungsten.

The clamping bodies particularly preferably consist of a material that is harder than the material of the at least one propulsion body, of the at least one securing element, and of the clamping rod.

As a result, the clamping bodies can be impressed into the clamping rod and the at least one propulsion body and the at least one securing element, but subsequently can still roll on the clamping rod.

Moreover, the invention can provide the inclined surface or edge of the cavity of the at least one propulsion body and/or the at least one securing element to consist(s) of metal or a metal-plastic composite.

It can be ensured by this means that the tightening clamping body can release again from the inclined surface or edge of the at least one propulsion body and/or of the at least one securing element.

Moreover, the invention can provide the at least one securing element to be arranged on the clamping rod ahead of or behind the at least one propulsion body with respect to the propulsion direction of the clamping rod. Preferably, the at least one securing element is arranged on the clamping rod between the at least one propulsion body and the adapter.

Preferred dispensing devices can provide the hardness of the clamping bodies and the clamping rod to be at least 50 HRC.

This allows multiple tightening and releasing of the clamping bodies on/off the clamping rod to be ensured.

A refinement of the present invention proposes the elastic elements, which push the at least one clamping body of the at least one propulsion body and the at least one clamping body of the at least one securing element onto the inclined surfaces and/or edges of the cavities of the at least one propulsion body and the at least one securing element, to be elastic bodies, which preferably close the cavities in the direction of the adapter.

This ensures that the clamping bodies can quickly block the at least one propulsion direction and the at least one securing element against the clamping rod since they are positioned suitably by the elastic bodies and are held in this position even against forces that may be acting.

According to a preferred embodiment of the present dispensing device, the forced closure between the at least one clamping body of the at least one propulsion body and the clamping rod, and the forced closure between the at least one clamping body of the at least one securing element and the clamping rod cannot be released at the same time, such that the clamping rod can be advanced only in one direction by the dispensing device.

By this simple constructive means, it is ensured that the clamping rod cannot be pushed backwards for reusing of the dispensing device. As a result, only sterile dispensing devices can be used, and unintentional contamination of the operating theatre or an infection of the patient are avoided.

The underlying objects of the invention are also met by a method for propelling a clamping rod with a rocker lever, in which at least one propulsion body touching against the clamping rod is moved in a first axial direction of the clamping rod by the action of a manual force on the rocker lever,

whereby the force of the motion pushes at least one clamping body against an inclined surface or edge of the cavity on the at least one propulsion body and the clamping rod, whereby the clamping body is arranged in a cavity of the at least one propulsion body, whereby the cavity is formed by the at least one propulsion body and the clamping rod, and whereby the clamping body touches the clamping rod in at least one spot or along a line, and whereby the pressure connects the at least one clamping body and the at least one propulsion body to the clamping rod in force-locking manner such that the clamping rod is moved along with the propulsion body,

whereby, as soon as the manual force acting on the rocker lever is reduced or ceases, the at least one propulsion body is pushed into the opposite second axial direction by a spring element, whereby the rocker lever is tilted again into the starting position, and whereby the at least one clamping body releases from the inclined surface or edge of the at least one propulsion body in the cavity due to the reverse action of force, and thus glides and/or rolls over the clamping rod,

whereby a reverse motion of the clamping rod into the second axial direction is blocked by at least one securing element touching against the clamping rod, whereby at least one clamping body of the securing element is pushed, by the reverse motion of the clamping rod, against an inclined surface or edge of the cavity on the at least one securing element and the clamping rod, whereby the clamping body is arranged in a cavity of the at least one securing element that is formed by the at least one securing element and the clamping rod, and whereby the clamping body touches the clamping rod in at least one spot or along a line, and the pressure acting on the clamping body connects the at least one clamping body and the at least one securing element to the clamping rod in force-locking manner such that the clamping rod does not move against the at least one securing element.

In this context, the invention can provide the at least one clamping body of the at least one securing element to be released from the inclined surface or edge of the cavity of the at least one securing element, and the clamping rod to thus be moved against the at least one securing element when the clamping rod is moved in the first axial direction.

This ensures that the clamping rod can be propelled without problems.

Moreover, the invention can provide the at least one clamping body of the at least one propulsion body and the at least one clamping body of the at least one securing element to be formed by spheres and/or rollers that roll on the clamping rod, at least to a minor extent.

Preferably, the spheres and/or rollers roll by at least 1° on the clamping rod.

Moreover, the invention can provide the inclined surface or edge of the cavity of the at least one propulsion body and/or the at least one securing element to have a wedge-shaped cross-section.

This allows a particularly simple design of the at least one propulsion body and/or the at least one securing element to be attained. The inclination of the surface or edge preferably is between 10° and 30°, particularly preferably between 15° and 25°, with respect to the clamping rod.

The invention can just as well provide the spring element to be compressed by the action of manual force when the rocker lever is being tilted.

By this means, the spring element is pre-tensioned for the subsequent restoration of the at least one propulsion body.

Moreover, the invention can provide for an elastic element to push the at least one clamping body of the at least one securing element from the direction of the adapter onto the inclined surface or edge of the cavity of the at least one securing element. The same applies with regard to the propulsion body as well.

This attains suitable positioning of the clamping body.

Methods according to the invention can also be characterised in that the motion is repeated multiply and the clamping rod is propelled stepwise in this context and a bone cement is expelled stepwise in this context from a cartridge that has previously been connected to a dispensing device by means of an adapter, whereby the dispensing device comprises the adapter, the rocker lever, the clamping rod, the at least one propulsion body, and the at least one securing element.

Finally, the invention can provide the method to be implemented through the use or by application of a dispensing device according to the invention.

The invention is based on finding, surprisingly, that the use of a securing element and a manually-driven propulsion body, which each comprise a mobile clamping body in a bevelled or wedge-shaped cavity and are arranged on a clamping rod such as to be mobile with respect to each other, allows a simple, but effective unidirectional propulsion of the clamping rod to be attained without the clamping rod possibly being driven backwards due to elastic forces of the PMMA cement cartridge. Moreover, provided the design is appropriate, the clamping bodies can be released from the cavities when the clamping rod is removed, which renders subsequent reassembly at least more difficult or even impossible. This restricts the re-usability such that the use of non-sterilised dispensing devices can be prevented. Concurrently, the entire design of the dispensing device can be implemented inexpensively such that the disposable product is not too expensive to manufacture. Furthermore, it is not possible to reuse the dispensing device, because the clamping rod can be advanced in only one direction.

It has also been found, surprisingly, that it is feasible through the use of the dispensing device according to the invention, despite the use of simple, inexpensive clamping rods and of inexpensive plastic parts, to dispense a polymethylmethacrylate bone cement dough without undesired retrograde motions of the dispensing plungers during the dispensation of polymethylmethacrylate bone cement dough from vacuum cementing systems. Moreover, it has been found that it is not possible to restore the dispensing device to its original condition without destroying it such that any re-use of the device after completed dispensation is excluded.

A dispensing device according to the invention can be composed of, for example, at least one manually-actuated rocker lever, a clamping rod, an adapter for the cement cartridge, and a housing with a handle. The dispensing device can be characterised, for example, in that

a) an axially shiftable propulsion body is arranged on the clamping rod and surrounds—at least regions of—the clamping rod;

b) the propulsion body possesses at least one cavity that is wedge-shaped or inclined in a longitudinal section, whereby the wedge shape or inclination is open towards the direction of motion of the clamping rod;

c) at least one sphere-shaped or roller-shaped clamping body is arranged in the cavity that is wedge-shaped or inclined in a longitudinal section, whereby the clamping body touches the clamping rod at least in spots or along a line;

d) an elastic element pushes the at least one clamping body into the cavity of the propulsion body that is wedge-shaped or inclined in a longitudinal section;

e) the rocker lever is arranged in the housing such that it can rotate such that one end of the rocker lever is pushed onto the axially shiftable propulsion body in the propulsion direction of the clamping rod, when the rocker lever is actuated;

f) a spring element pushes on the propulsion body in opposite direction with respect to the propulsion direction of the clamping rod;

g) the housing has a securing element connected to it that surrounds—at least regions of—the clamping rod, whereby the securing element possesses at least one wedge-shaped or inclined cavity that is open towards the propulsion direction of the clamping rod;

h) at least one sphere-shaped or roller-shaped clamping body that is arranged in the cavity that is wedge-shaped or inclined in a longitudinal section, whereby the sphere-shaped or roller-shaped clamping body touches the clamping rod at least in spots or along a line; and in that

i) at least one elastic element pushes the clamping body into the wedge-shaped or inclined cavity of the securing element.

The housing and the adapter for the cement cartridge preferably consist of plastic material. The clamping rod is made of metal, preferably of steel. The rocker lever consists either of plastic material, and aluminium alloys or steel.

The rocker lever is preferably designed as a lever system in accordance with EP 0 326 551 A1. Said lever system uses a manually-actuated lever parallelogram that enables forceful actuation of the lever by the index finger or middle finger.

The feed-through of the propulsion body is preferably designed as a tube. By this means, tipping and tilting of the propulsion body on the clamping rod is excluded.

The cavity of the propulsion body and also of the securing element, which is wedge-shaped or inclined in a longitudinal section, is preferably designed as a funnel. In addition, it is also feasible to design the cavity as sections of a funnel. It is feasible just as well to design the cavity to be a wedge-shaped prism.

The clamping bodies are formed from metal and/or ceramics. Preferably, the clamping bodies consist of steel or tungsten. In addition, it is feasible just as well that the clamping bodies are made from hard ceramic materials, such as silicon carbide, tungsten carbide or boron carbide.

The cavity, which is wedge-shaped or inclined in a longitudinal section, consists of metal or of a metal-plastic composite. It is important in this context that the clamping bodies are supported in a hard abutment such that the clamping bodies cannot deform the propulsion body and the securing element when they clamp the propulsion body and the securing element. Deformation of the propulsion body and of the securing element might lead to blockage of the motion of the clamping rod.

However, smaller or minor plastic deformations are no issue as long as they do not impair the function of the dispensing device.

The securing element can be arranged on the clamping rod either before or after the propulsion body, with respect to the propulsion direction.

The clamping bodies and the clamping rod have a hardness of at least 50 HRC.

Preferably, the hardness is at least 56 HRC. The term “HRC” shall be understood to refer to the Rockwell hardness according to scale C according to DIN EN ISO 6508-1.

A method according to the invention for dispensing polymethylmethacrylate bone cement by means of a dispensing device according to the invention can be characterised, for example, in that

in a step a), the rocker lever is moved manually against the propulsion body that is situated in a starting position, whereby the propulsion body, by clamping with the at least one clamping body, is clamped against the clamping rod and the cavity of the clamping rod that is wedge-shaped in a longitudinal section, and moves in the propulsion direction, whereby concurrently the spring element is being compressed,

in a step be b), once the propulsion motion of the rocker lever is completed, the propulsion body is pushed back into the starting position by the compressed spring element on the clamping rod,

in a step c), the securing element is secured against moving in a direction opposite to the propulsion direction by clamping of the at least one clamping body against the clamping rod and the cavity that is wedge-shaped in a longitudinal section, and in that steps a through c are repeated.

### BRIEF DESCRIPTION OF THE DRAWINGS

Further exemplary embodiments of the invention shall be illustrated in the following on the basis of six schematic figures, though without limiting the scope of the invention. In the figures:

FIG. 1: shows a schematic cross-sectional view of a dispensing device according to the invention;

FIG. 2: shows a schematic perspective view of the dispensing device according to FIG. 1;

FIG. 3: shows a schematic perspective side view of the dispensing device according to FIGS. 1 and 2;

FIG. 4: shows a schematic perspective view, from an angle from below, of the dispensing device according to FIGS. 1 to 3;

FIG. 5: shows a schematic top view, from above, of the dispensing device according to FIGS. 1 to 4; and

FIG. 6: shows a schematic perspective cross-sectional view of a portion of an alternative dispensing device of the present invention.

In the Figures, the same reference numbers are used also for different embodiments.

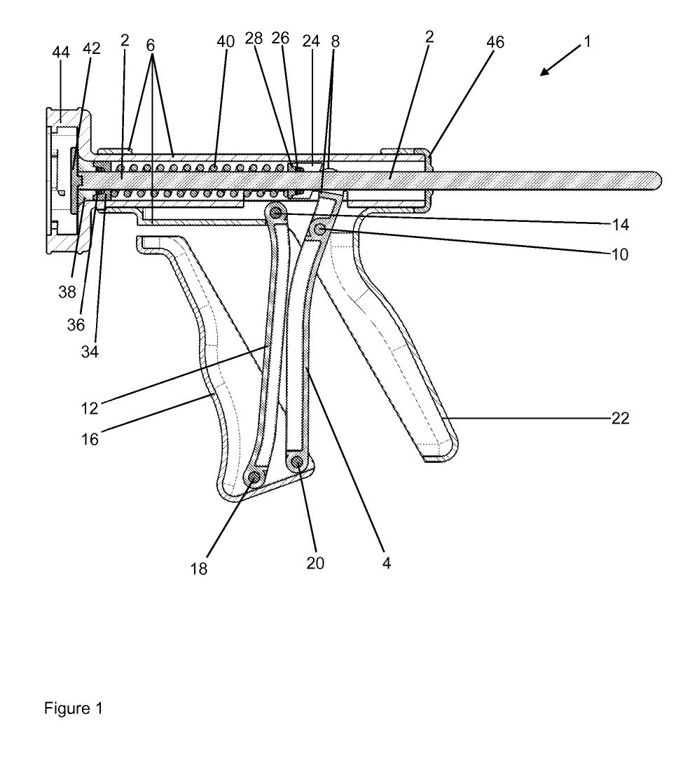

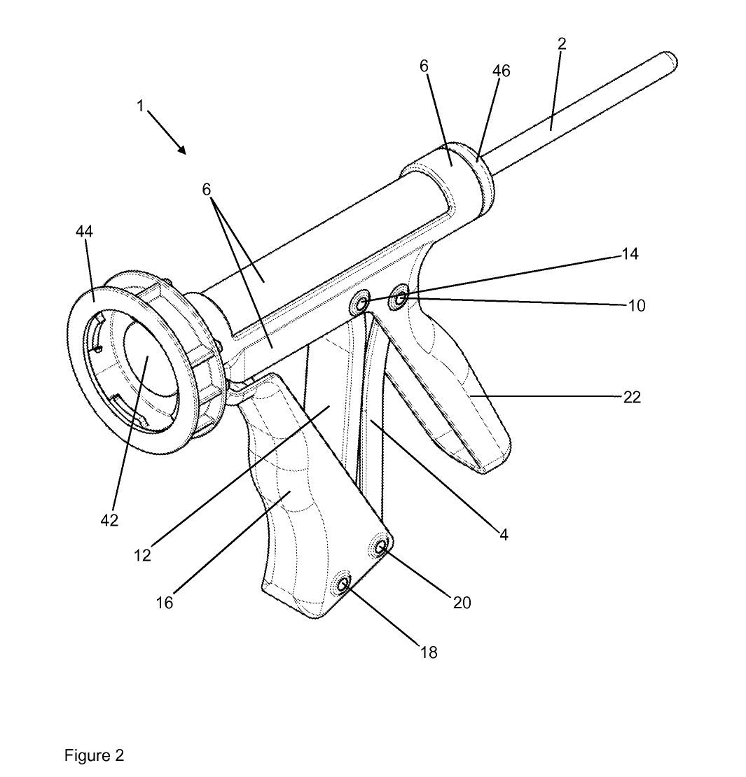

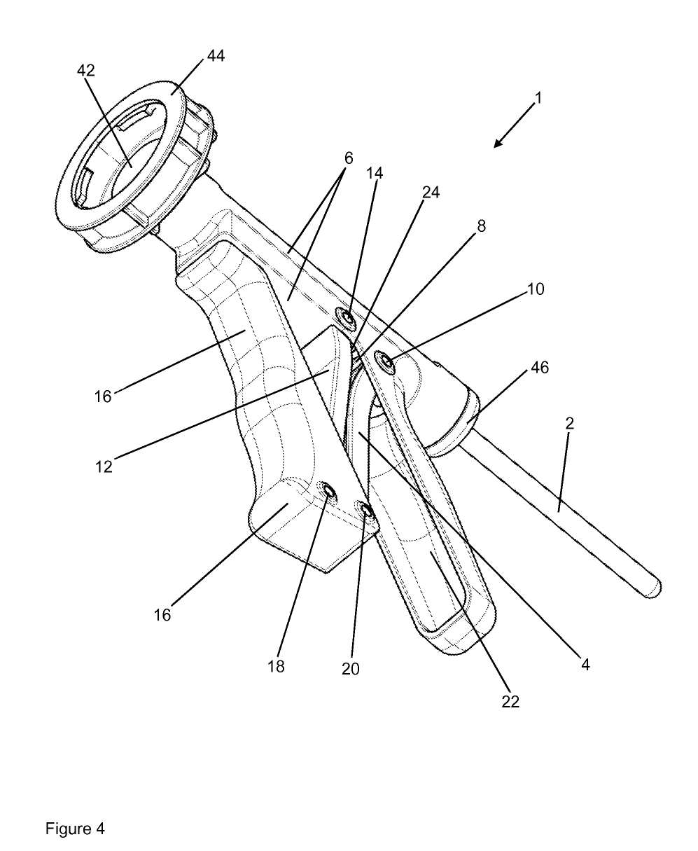

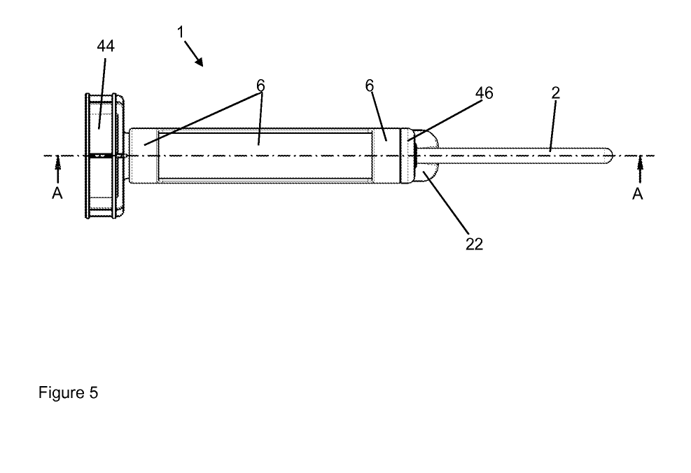

FIG. 1 shows a dispensing device 1 with a clamping rod 2 that can be propelled forward (left in FIG. 1). FIGS. 2 to 4 show related perspective views. A top view of the dispensing device 1 is shown in FIG. 5. The cross-section shown in FIG. 1 is identified in FIG. 5 through line-dot line A. Accordingly, the cross-sectional view according to FIG. 1 corresponds to the section along the line A-A in FIG. 5.

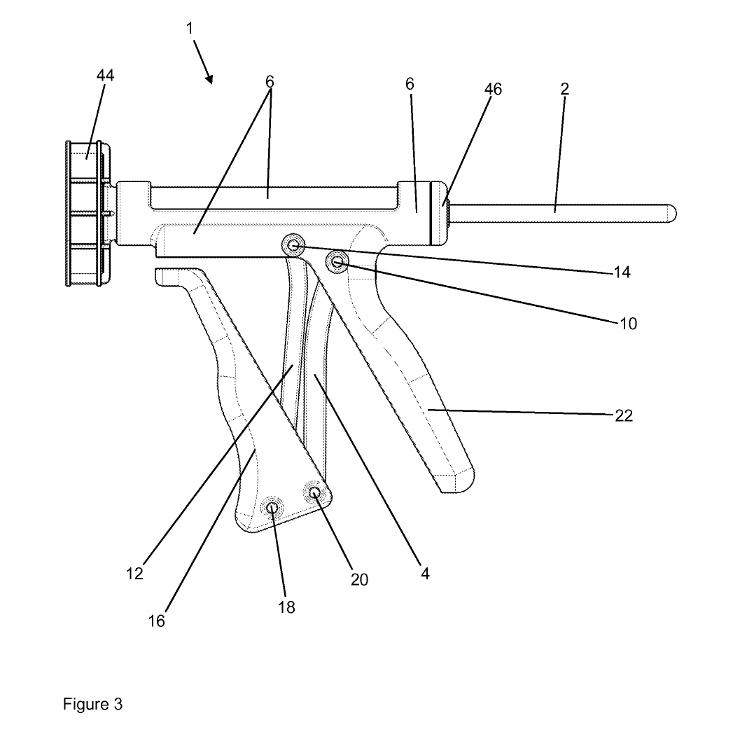

The clamping rod 2 can be moved by means of a rocker lever 4 made of a stable plastic material or, alternatively, of steel. The dispensing device 1 is largely surrounded by a multi-part housing 6 made of plastics such that the internal design of the dispensing device 1 is not exposed. The housing parts 6 can be manufactured as injection moulding parts. The rocker lever 4 terminates in a pivoting head 8 by means of which the motion of the rocker lever 4 is transmitted into the inside of the housing 6 of the dispensing device 1. For this purpose, the rocker lever 4 is connected to the housing 6 by means of an axis 10. When the rocker lever 4 is rotated about the axis 10 (counter-clockwise in the top view of the cross-section according to FIG. 1 and the side view according to FIG. 3), the head 8 of the rocker lever 4 is pushed forward at great force due to the leverage effect (towards the left in FIGS. 1, 3, and 5).

The rocking lever 4 is operated by moving a strut 12 that is connected to the housing 6 by means of an axis 14 and to a trigger 16 by means of an axis 18. The rocker lever 4 is also connected to the trigger 16, namely by means of an axis 20. Due to this design, the trigger 16 can be moved parallel to a handle 22. Being a part of the housing 6, the handle 22 is made of plastics. Due to the design involving the rocker lever 4 and the strut 12 as well as their connections/axes 10, 14, 18, 20 to the housing 6 and the trigger 16, the full height of the trigger 16 can be used to exert a pressure onto the rocker lever 4. By this means, the rocker lever 4 can be operated with the full force of the entire hand in particular including the force of the index finger and middle finger, whereby the handle 22 is being held by the same hand.

Accordingly, the entire dispensing device 1 is easy to hold and operate with one hand. A design of this type is described in detail in EP 0 326 551 A1 as well.

The head 8 of the rocker lever 4 is used to propel a propulsion body 24 forward (shifting it towards the left in FIGS. 1, 3, and 5). The propulsion body 24 surrounds the clamping rod 2 and comprises, on the inside, a recess that forms a cavity together with the clamping rod 2. Multiple spheres 26 made of steel or ceramic are arranged in the cavity as clamping bodies 26 that touch against the clamping rod 2. The cavity and/or the recess of the propulsion body 24 has a boundary surface that is inclined forward (towards the left in FIGS. 1, 3, and 5) and is provided in the way of a funnel such as to also be circumferential about the clamping rod 2. A multitude of spheres 26 are arranged as clamping bodies 26 in the way of a ball bearing in the cavity that is bounded by the funnel and the clamping rod 2 as well as a closure 28 on the front side (on the left in FIGS. 1, 3, and 5). The closure 28 is elastic and pushes the spheres 26 lightly against the inclined wall of the cavity. The funnel wall is is inclined by approximately 20° with respect to the axis of the clamping rod 2.

When the head 8 of the rocker lever 4 pushes onto the propulsion body 24 due to a rotation about the axis 10, the spheres 26 are pressed against the inclined wall of the cavity and the clamping rod 2. As a result, the spheres 26 and/or the clamping bodies 26 block and the propulsion body 24 and the clamping rod 2 become lodged and/or wedged against each other. Since the contact surface (contact spots) of the spheres 26 on the clamping rod 2 and/or on the wall of the cavity is small, a very large pressure arises on the contact surfaces that is sufficient to prevent the propulsion body 24 from being pushed forward on the clamping rod 2.

The front side of the dispensing device 1 (on the left in FIGS. 1, 3, and 5) has a securing element 34 provided in it, which is identical in design to the propulsion body 24, whereby the securing element 34 is connected firmly to the housing 6, whereas the propulsion body 24 is supported as in a bearing to be mobile in the housing 6.

The securing element 34 surrounds the clamping rod 2 and comprises, on the inside, a recess that forms a cavity together with the clamping rod 2. Multiple spheres 36 made of steel or ceramic are arranged in the cavity as clamping bodies 36 that touch against the clamping rod 2. The cavity and/or the recess of the securing element 34 has a boundary surface that is inclined forward (towards the left in FIGS. 1, 3, and 5) and is provided in the way of a funnel such as to also be circumferential about the clamping rod 2. A multitude of spheres 36 are arranged as clamping bodies 36 in the way of a ball bearing in the cavity that is bounded by the funnel and the clamping rod 2 as well as a closure 38 on the front side (on the left in FIGS. 1, 3, and 5). The closure 38 is elastic and pushes the spheres 36 lightly against the inclined wall of the cavity. The funnel wall is inclined by approximately 20° with respect to the axis of the clamping rod 2.

When the clamping rod 2 with the rocker lever 4 and the propulsion body 24 is propelled forward, the clamping rod 2 slides through the securing element 34 since the spheres 36 can easily roll on the clamping rod 2 during this motion due to funnel wall being inclined. Concurrently, an elastic spring 40 that is arranged between the propulsion body 24 and the securing element 34 in the housing 6 is being tensioned and/or compressed elastically.

When the force acting on the rocker lever 4 lessens or ceases, the tensioned spring 40 pushes the propulsion body 24 in the opposite direction towards the back (towards the right in FIGS. 1, 3, and 5). The spheres 26 of the propulsion body 24 can easily roll on the rod 2 during this motion, because the funnel wall of the cavity is inclined. As a result, the propulsion body 24 can be shifted on the clamping rod 2.

Concurrently, is not feasible to push the clamping rod 2 back towards the rear into the housing 6 (towards the right in FIGS. 1, 3, and 5), since this motion is blocked by the clamping bodies 36 and/or the spheres 36 due to the funnel of the securing element 34 being inclined. Accordingly, if the extrusion of bone cement from the cartridge (not shown) is associated with elastic forces exerting a counter-pressure onto the clamping rod 2, the clamping rod 2 cannot be pushed back into the housing 6.

On the front side, a punch 42 is attached to the clamping rod 2 and is intended for propelling a conveying plunger of a cartridge (not shown). An adapter 44 with a bayonet closure for connecting a cement cartridge is situated on the front side of the dispensing device 1. The cement cartridge (not shown) can be attached to the adapter 44, whereby the bottom of the attached cartridge contains the conveying plunger that can be pushed into the cartridge by means of the punch 42. When the clamping rod 2 is propelled, the punch 42 pushes the conveying plunger into the cartridge, whereby the cartridge content (for example a medical PMMA bone cement) is pushed from the cartridge through a cartridge opening that is situated opposite from the conveying plunger.

The dispensing device is closed on the back by a cap 46. The cap 46 comprises a feed-through for the clamping rod 2 and can be considered to be a part of the housing 6.

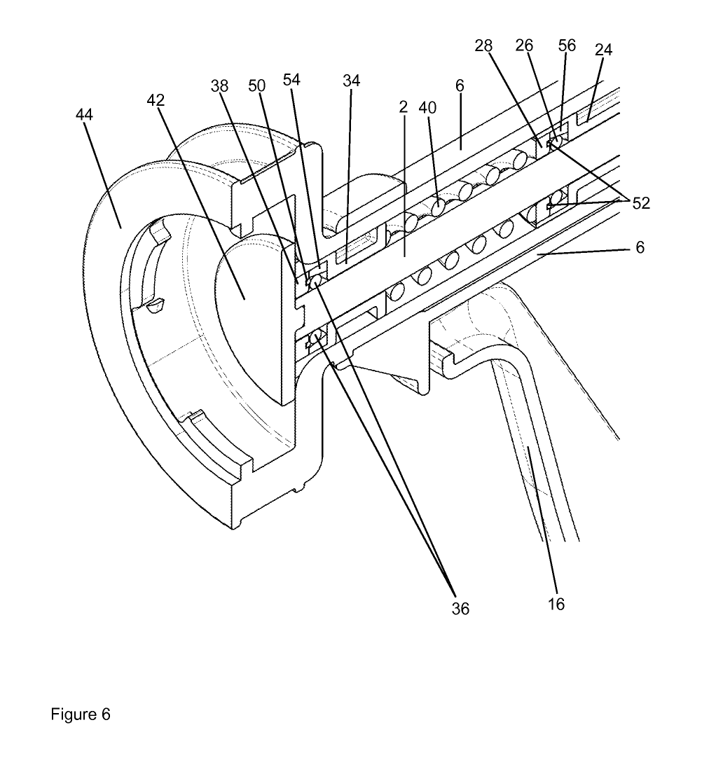

FIG. 6 shows a schematic perspective cross-sectional view of a portion of an alternative dispensing device of the present invention. The dispensing device corresponds to the discussion in connection with FIGS. 1 to 5 with the exception of some details of the propulsion body 24 and the securing element 34.

A rod 2 is mounted in the dispensing device to be movable in longitudinal direction. The dispensing device comprises a housing 6 made of plastic, in which a clamping rod 2 is movably mounted. This dispensing device is actuated by a trigger 16, which is positioned in the region of a handle (not shown) at the bottom side (in FIG. 6 below) of the dispensing device in analogy to FIGS. 1 to 5, and which is actuated via a lever parallelogram in analogy thereto. For advancing the rod 2, a propulsion body 24 is provided having a cavity in which spheres 26 or rolls 26 are arranged. The cavity of the propulsion body is closed by a closure 28 on the front side (in FIG. 6 on the left side). Analogously, a securing element 34 is constructed, which also forms a cavity together with the rod 2, in which spheres 26 and rolls 26 are arranged. The securing element 34 is firmly attached to the housing 6, while the propulsion body 24 is movably arranged in the housing 6. The cavity of the securing element 34 is also closed by a closure 38 on the front side.

An elastic spring 40 made of steel is arranged between the propulsion body 24 and the securing element 34, the elastic spring pushing the propulsion body 24 away from the securing element 34 (backward—in FIG. 6 to the right side). On the front side, the rod 2 ends as a punch 42, which is situated in an adapter 44 having a bayonet connector.

Within the closure 38, there is a surrounding groove receiving an elastic O-ring 50 made of rubber. In analogy, a surrounding groove is arranged within the cavity 28 receiving an elastic O-ring 52 made of rubber. Further, a cone 54 is provided within the cavity of the propulsion body 24 as an inserted part, and a cone 56 is provided within the cavity of the propulsion body 24. The cones 54, 56 form inclined walls, against which the spheres 26, 36 are pressed by the elastic O-rings 50, 52. The inclination of the cone 56 of the propulsion body 24 presses the spheres 26 against the rod 2, when the propulsion body 24 is advanced in direction of the security element 34, such that the rod 2 is moved together with the propulsion body 24. The inclination of the cone 54 of the securing element 34 presses the spheres 36 against the rod 2, when the rod 2 is moved backwards (in FIG. 6 to the right side). By this means, a movement of the rod 2 together with the propulsion body 24 backwards is prevented (in FIG. 6 to the right side).

The propulsion body 24 is advanced forward in direction of the securing element 34 by the trigger 16. This attains pushing the rod 2 forward and biasing the spring 40. In releasing the trigger 16, the propulsion body 24 is pushed again away from the securing element 34. The securing elements prevents a movement of the rod 2 backwards.

The construction of both embodiments according to FIGS. 1 to 6 allows moving the rod 2 only in forward direction, while moving the rod 2 backwards is prevented. There is no possibility provided to move back the rod 2 into the starting position and reuse of the dispensing device is excluded. The features of the invention disclosed in the preceding description and in the claims, figures, and exemplary embodiments, can be essential for the implementation of the various embodiments of the invention both alone and in any combination.

### LIST OF REFERENCE NUMBERS

1 Dispensing device
2 Clamping rod
4 Rocker lever
6 Housing
8 Head of the rocker lever
10 Axis
12 Strut
14 Axis
16 Trigger
18 Axis
20 Axis
22 Handle
24 Propulsion body
26 Sphere/clamping body
28 Closure
34 Securing element
36 Sphere/clamping body
38 Closure
40 Spring
42 Punch
44 Adapter/bayonet connector
46 Cap
50 O-ring
52 O-ring
54 cone/inserted part
56 cone/inserted part

1. Dispensing device (1) for cement cartridges for polymethylmethacrylate bone cements comprising at least one manually actuatable rocker lever (4), an adapter (44) for a cement cartridge, and a clamping rod (2) that can be propelled in the direction of the adapter (44),
whereby at least one propulsion body (24) is arranged to be touching against the clamping rod (2) and is shiftable in axial direction with respect to the axis of the clamping rod (2),
whereby the at least one propulsion body (24) touching against the clamping rod (2) forms at least one cavity together with the clamping rod (2), whereby the cavity comprises a surface or edge that is inclined in the propulsion direction of the clamping rod (2) such that the cavity widens in the direction of the adapter in the region of said surface or edge,
whereby at least one sphere-shaped or roller-shaped clamping body (26) is arranged in the cavity of the at least one propulsion body (24), whereby the at least one clamping body (26) of the at least one propulsion body (24) touches the clamping rod (2) in at least one spot or along a line,
whereby an elastic element (28 52) pushes the at least one clamping body (26) of the at least one propulsion body (24) from the direction of the adapter (44) onto the inclined surface or edge of the cavity of the at least one propulsion body (24),
whereby the rocker lever (4) is supported as in a bearing such that it can rotate with respect to the clamping rod (2), such that, upon actuation of the rocker lever (4), an end (8) of the rocker lever (4) pushes onto the at least one axially shiftable propulsion body (24) in propulsion direction of the clamping rod (2),
whereby a spring element (40) pushes onto the at least one propulsion body (24) opposite to the propulsion direction of the clamping rod (2),
at least one securing element (34) touches against the clamping rod (2) against which the propulsion body (24) is supported such as to be mobile,
whereby the at least one securing element (34) touching against the clamping rod (2) forms at least one cavity together with the clamping rod (2), whereby the cavity comprises a surface or edge that is inclined with respect to the propulsion direction of the clamping rod (2) such that the cavity widens in the direction of the adapter (44) in the region of said surface or edge,
whereby at least one sphere-shaped or roller-shaped clamping body (36) is arranged in the cavity of the at least one securing element (34), whereby the at least one clamping body (36) of the at least one securing element (34) touches the clamping rod (2) in at least one spot or along a line,
whereby an elastic element (38, 50) pushes the at least one clamping body (36) of the at least one securing element (34) from the direction of the adapter (44) onto the inclined surface or edge of the cavity of the at least one securing element (34).

2. Dispensing device (1) according to claim 1, wherein
the dispensing device (1) comprises a housing (6) with a handle (22), whereby the at least one securing element (34) is firmly connected to the housing (6) and the at least one propulsion body (24) is supported as in a bearing against the housing (6) such as to be mobile.

3. Dispensing device (1) according to claim 1, wherein
the at least one propulsion body (24) surrounds the clamping rod (2), at least in regions thereof.

4. Dispensing device (1) according to claim 3, wherein
the at least one propulsion body (24) comprises a feed-through that optionally is provided as a tube.

5. Dispensing device (1) according to claim 1, wherein
multiple axially shiftable propulsion bodies (24) touch against the clamping rod (2) from different directions.

6. Dispensing device (1) according to claim 1, wherein
the inclined surface is shaped appropriately such that the at least one propulsion body (24) and the at least one securing element (34) comprises at least one cavity that is inclined, in longitudinal section, in the direction of the adapter (44), comprises a wedge-shaped cavity, whereby the wedge shape is open in the direction of the adapter (44).

7. Dispensing device (1) according to claim 1, wherein
at least one sphere-shaped or roller-shaped clamping body (26, 36) is arranged or supported as in a bearing such that it can rotate about at least one axis in the cavity of the at least one propulsion body (24) and/or the at least one securing element (34).

8. Dispensing device (1) according to claim 1, wherein
the cavity formed by the at least one propulsion body (24) and the clamping rod (2) and/or the cavity formed by the at least one securing element (34) and the clamping rod (2) is or are formed as a funnel, whereby the funnel surrounds the clamping rod (2).

9. Dispensing device (1) according to claim 1, wherein
the clamping bodies (26, 36) are made from ceramics or metal.

10. Dispensing device (1) according to claim 1, wherein
the inclined surface or edge of the cavity of the at least one propulsion body (24) and/or the at least one securing element (34) consist(s) of metal or a metal-plastic composite.

11. Dispensing device (1) according to claim 1, wherein
the at least one securing element (34) is arranged on the clamping rod (2) ahead of or behind the at least one propulsion body (24) with respect to the propulsion direction of the clamping rod (2).

12. Dispensing device (1) according to claim 1, wherein
the clamping bodies (36) and the clamping rod (2) have a hardness of at least 50 HRC.

13. Dispensing device (1) according to claim 1, wherein
the elastic elements (28, 52, 38, 50), which push the at least one clamping body (26) of the at least one propulsion body (24) and the at least one clamping body (36) of the at least one securing element (34) onto the inclined surfaces and/or edges of the cavities of the at least one propulsion body (24) and the at least one securing element (34), are elastic bodies, which optionally close the cavities in the direction of the adapter (44).

14. Dispensing device (1) according to claim 1, wherein
the forced closure between the at least one clamping body (26) of the at least one propulsion body (24) and the clamping rod (2) and the forced closure between the at least one clamping body (36) of the at least one securing element (34) and the clamping rod (2) are not releasable at the same time, such that the clamping rod (2) is advancable only in one direction by the dispensing device.

15. Method for propelling a clamping rod (2) with a rocker lever (4), in which at least one propulsion body (24) touching against the clamping rod (2) is moved in a first axial direction of the clamping rod (2) by the action of a manual force,
whereby the force of the motion pushes at least one clamping body (26) against an inclined surface or edge of the cavity on the at least one propulsion body (24) and the clamping rod (2), whereby the clamping body (26) is arranged in a cavity of the at least one propulsion body (24), whereby the cavity is formed by the at least one propulsion body (24) and the clamping rod (2), and whereby the clamping body (26) touches the clamping rod (2) in at least one spot or along a line, and whereby the pressure connects the at least one clamping body (26) and the at least one propulsion body (24) to the clamping rod (2) in force-locking manner such that the clamping rod (2) is moved along with the propulsion body (24),
whereby, as soon as the manual force acting on the rocker lever (4) is reduced or ceases, the at least one propulsion body (24) is pushed into the opposite second axial direction by a spring element (40), whereby the rocker lever (4) is tilted again into the starting position, and whereby the at least one clamping body (26) releases from the inclined surface or edge of the at least one propulsion body (24) in the cavity due to the reverse action of force, and thus glides and/or rolls over the clamping rod (2),
whereby a reverse motion of the clamping rod (2) into the second axial direction is blocked by at least one securing element (34) touching against the clamping rod (2), whereby at least one clamping body (36) of the securing element (34) is pushed, by the reverse motion of the clamping rod (2), against an inclined surface or edge of the cavity on the at least one securing element (34) and the clamping rod (2), whereby the clamping body (36) is arranged in a cavity of the at least one securing element (34) that is formed by the at least one securing element (34) and the clamping rod (2), and whereby the clamping body (36) touches the clamping rod (2) in at least one spot or along a line, and the pressure acting on the clamping body (36) connects the at least one clamping body (36) and the at least one securing element (34) to the clamping rod (2) in force-locking manner such that the clamping rod (2) does not move against the at least one securing element.

16. Method according to claim 15, wherein
the at least one clamping body (36) of the at least one securing element (34) is released from the inclined surface or edge of the cavity of the at least one securing element (34), and the clamping rod (2) is thus moved against the at least one securing element (34) when the clamping rod (2) is moved in the first axial direction.

17. Method according to claim 16, wherein
the at least one clamping body (26) of the at least one propulsion body (24) and the at least one clamping body (36) of the at least one securing element (34) are formed by spheres and/or rollers that roll on the clamping rod (2), at least to a minor extent.

18. Method according to claim 17, wherein
the inclined surface or edge of the cavity of the at least one propulsion body (24) and/or the at least one securing element (34) has/have a wedge-shaped cross-section.

19. Method according to claim 15, wherein
the spring element (40) is compressed by the action of manual force when the rocker lever (4) is being tilted.

20. Method according to claim 15, wherein
an elastic element (38) pushes the at least one clamping body (36) of the at least one securing element (34) from the direction of the adapter (44) onto the inclined surface or edge of the cavity of the at least one securing element (34).

21. Method according to claim 15, wherein
the motion is repeated multiply and the clamping rod (2) is propelled stepwise in this context and a bone cement is expelled stepwise in this context from a cartridge that has previously been connected to a dispensing device (1) by means of an adapter (44), whereby the dispensing device (1) comprises the adapter (44), the rocker lever (4), the clamping rod (2), the at least one propulsion body (24), and the at least one securing element (34).

22. Method according to claim 15, wherein
the method is performed using a dispensing device (1) according to claim 1.
